(19) United States
(12) Patent Application Publication
(10) Pub. No.: US 2024/0148279 A1
(43) Pub. Date: May 9, 2024

(54) HIP KNEE RANGE OF MOTION MEASUREMENT DEVICE

(71) Applicant: Imam Abdulrahman Bin Faisal University, Dammam (SA)

(72) Inventors: Shibili Nuhmani Aliyangat VAKERI, Dammam (SA); Ali Muteb Y. ALSHAMI, Dammam (SA)

(73) Assignee: Imam Abdulrahman Bin Faisal University, Dammam (SA)

(21) Appl. No.: 17/981,063

(22) Filed: Nov. 4, 2022

(51) Int. Cl. A61B 5/11; A61B 5/00; A61B 5/107

(52) U.S. Cl. CPC A61B 5/1121; A61B 5/1071; A61B 5/4571; A61B 5/4585; A61B 5/6828; A61B 5/6831; A61B 2505/09

(57) ABSTRACT

A hip knee range of motion measurement device includes a first movable arm, a second movable arm connected to the first movable arm via a first joint, and a third movable arm connected to the second movable arm via a second joint. The device further includes a first dial attached to the second movable arm and a second dial attached to the third movable arm, each configured to measure a joint angle of the patient. The first movable arm remains longitudinally oriented with a horizontal axis of a pelvis of a patient, the second movable arm remains in axial alignment to a horizontal axis of a femur of the patient, and the third movable arm remains in axial alignment to a horizontal axis of a tibia of the patient.

### BACKGROUND

### Technical Field

The present disclosure relates to a dynamic joint measuring techniques and more particularly relates to range of motion measurement device for hip and knee joints simultaneously.

### Discussion of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present invention.

Several studies have reported relation between hamstring muscle flexibility and lower limb injuries. Tightness of hamstrings may be a result of faulty posture, movement abnormalities, and sports injuries. Hamstring injuries have a high occurrence and recurrence rate in the athletic population. Hamstring muscle stretching is known in sports to prevent the hamstring injuries and is an essential component in warmup activities in athletics. The hamstring is a two joint muscle that extends between hip and knee joints, and thus builds a relation between hip flexion range of motion (ROM) and knee extension ROM. In addition, there are several objective examination and treatment techniques where simultaneous movement of the hip and knee joints must be performed. In some of these techniques, either the hip or knee joint has to be kept in a certain degree of range of motion. Thus, simultaneous measurement of hip and knee joint angles is important for these techniques. For example, active knee extension (AKE) and passive knee extension (PKE) tests are considered reliable and widely used objective measurement techniques to assess the length of the hamstring muscles. These tests are generally performed by relatively inexperienced testers or by athletes themselves when working in pairs as part of a general exercise screening program. The AKE and PKE tests measure an angle of knee flexion (known as popliteal angle) with the hip stabilized in 90 degrees of flexion. Stabilization of the hip joint, in 90-degree position or other degrees, and proper placement of a measuring instrument (such as goniometer/inclinometer) are important in achieving accuracy of these tests. However, no clinical apparatus is available for these tests. Therefore, more than one assessor is required to perform the tests, and the assessors may not be able to obtain accurate range of joint motion.

Straight leg raise (SLR) is another technique that can be used as an examination test or a treatment technique, particularly with peripheral neural sensitization affecting lower limb. During SLR, simultaneous movement of more than one joint is required. For example, the hip joint needs to be stabilized in a certain degree of flexion while moving other joints such as the knee as a sensitizing movement.

U.S. Pat. No. 10,010,714B2 describes a system for delivering stimulation therapy or relief to a patient. The system includes a stimulation electrode pair coupled to a good that can couple to a patient to deliver stimulation controlled through a wired or wireless link; a controller that can generate and apply a stimulation pulse to the stimulation electrode pair based on the stimulation program and the signal or data; a sensor coupled to the controller and the good; and a user interface enabling a user to remotely view or exchange information through the link, and to monitor and set or reconfigure the stimulation pulse. However, a process followed using the system of this reference is relatively cumbersome and, and the measurements thus obtained may be inconsistent.

U.S. Pat. No. 4,485,825A describes an instrument for measuring positions and displacements of portions of a human body, including a base plate and a belt or suction cup for detachably securing the base plate to a portion of a human body. A dip circle and a north-south-seeking compass are carried by a housing, the dip circle and compass being mounted on the housing for pivotal movement. The base plate has a scale thereon, and an indicator for indicating on the scale the degree of rotation of a holder relative to the base plate. There is also a scale for indicating the degree of rotation of the dip circle and compass relative to the housing. However, this reference does not describe the movable arms, the joint, and the movable straps of the present disclosure.

U.S. Pat. No. 4,883,069A describes a three-plane goniometer or angle measuring apparatus that includes three small potentiometers which are closely spaced together to measure rotation of the knee about three different axes. The unit is primarily mounted on a cuff on the outside of the thigh. The mounting assembly for the three plane goniometer includes a curved yoke where the ends of the yoke curve from the front to the rear, with the goniometer unit being held between the ends of the rearwardly extending arms of the yoke. However, the apparatus of this reference is complex and does not include parts with simple construction as described in the present disclosure.

U.S. Pat. No. 8,282,579B2 describes a goniometer for use in measuring the range of motion of a patient. The goniometer includes a first arm and a second arm that are pivotally attached. A scale is present and is used in determining the angular orientation between the first arm and the second arm. Also, a level member is included and is carried by one of the first arm and second arm.

Each of the aforementioned patent reference suffers from one or more drawbacks hindering their adoption, specifically none of the above-mentioned instruments can measure the range of motion of the hip and knee joints simultaneously. Accordingly, it is one object of the present disclosure to provide a device that can accurately measure values from the tests and techniques where simultaneous hip and knee movement is required, which makes sure that the hip is stabilized in 90 degrees of flexion and measure the popliteal angle by a single assessor.

### SUMMARY

According to an aspect of the present disclosure, a hip knee range of motion measurement device is provided. The device includes a first movable arm, a second movable arm, and a third movable arm, where each movable arm has a first end and a second end. The first, second, and third movable arms are coplanar. The device also includes a first joint attached to the first end of the first movable arm and a second join attached to a second end of the second movable arm. The first end of the second movable arm is attached to the first joint and the first end of the third movable arm is attached to the second joint. The device further includes a first dial attached to the second movable arm, a second dial attached to the third movable arm, a first movable strap attached to the first movable arm, a second movable strap attached to the second movable arm, and a third movable strap attached to the third movable arm. The first movable arm is configured to remain longitudinally oriented with a horizontal axis of a pelvis of a clinical patient with the first movable strap, the second movable arm is configured to remain in axial alignment to a horizontal axis of a femur of the clinical patient with the second movable strap, and the third movable arm is configured to remain in axial alignment to a horizontal axis of a tibia of the clinical patient with the third movable strap. Further, the first joint is configured to be positioned adjacent to a hip joint of the clinical patient and is configured to remain longitudinally oriented with a horizontal axis of a hip of the clinical patient and the second joint is configured to be positioned adjacent to a knee joint of the clinical patient. The first and the second dials are configured to measure a joint angle of the clinical patient.

In some embodiments, the device is configured to provide range of motion values of both the hip and a knee of the clinical patient simultaneously.

In some embodiments, the first, second, and third movable arms are configured to stabilize the hip joint of the clinical patient while measuring a popliteal angle of the clinical patient.

In some embodiments, the first dial is configured to measure the hip angle of the clinical patient at hip joint angles ranging from 0 degrees to 360 degrees.

In some embodiments, the second dial is configured to measure a popliteal angle of the clinical patient at the knee joint ranging from 0 degrees to 360 degrees.

In some embodiments, the first and second joint includes adjustable breaks.

In some embodiments, the first dial is equally spaced between the first end and the second end of the second movable arm.

In some embodiments, the second dial is equally spaced between the first end and the second end of the third movable arm.

In some embodiments, the second arm is configured to be adjustable in a range of from 0 degrees to 360 degrees.

In some embodiments, each of the first joint and the second joint is rotatable 360 degrees.

In some embodiments, a length of the first movable arm, a length of the second movable arm, and a length of the third movable arm are each slidably adjustable by half the total length of the first movable arm, the second movable arm, and the third movable arm respectfully.

These and other aspects of non-limiting embodiments of the present disclosure will become apparent to those skilled in the art upon review of the following description of specific non-limiting embodiments of the disclosure in conjunction with the accompanying drawings.

### BRIEF DESCRIPTION OF THE DRAWINGS

A better understanding of embodiments of the present disclosure (including alternatives and/or variations thereof) may be obtained with reference to the detailed description of the embodiments along with the following drawings, in which:

FIG. 1 illustrates a perspective view of a hip knee range of motion measurement device, according to an aspect of the present disclosure; and

FIG. 2 illustrates a clinical patient wearing the hip knee range of motion measurement device, according to an aspect of the present disclosure.

### DETAILED DESCRIPTION

In the following description, it is understood that other embodiments may be utilized, and structural and operational changes may be made without departure from the scope of the present embodiments disclosed herein.

Reference will now be made in detail to specific embodiments or features, examples of which are illustrated in the accompanying drawings. Wherever possible, corresponding, or similar reference numbers will be used throughout the drawings to refer to the same or corresponding parts. Moreover, references to various elements described herein, are made collectively or individually when there may be more than one element of the same type. However, such references are merely exemplary in nature. It may be noted that any reference to elements in the singular may also be construed to relate to the plural and vice-versa without limiting the scope of the disclosure to the exact number or type of such elements unless set forth explicitly in the appended claims.

In the drawings, like reference numerals designate identical or corresponding parts throughout the several views. Further, as us herein, the words “a,” “an” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

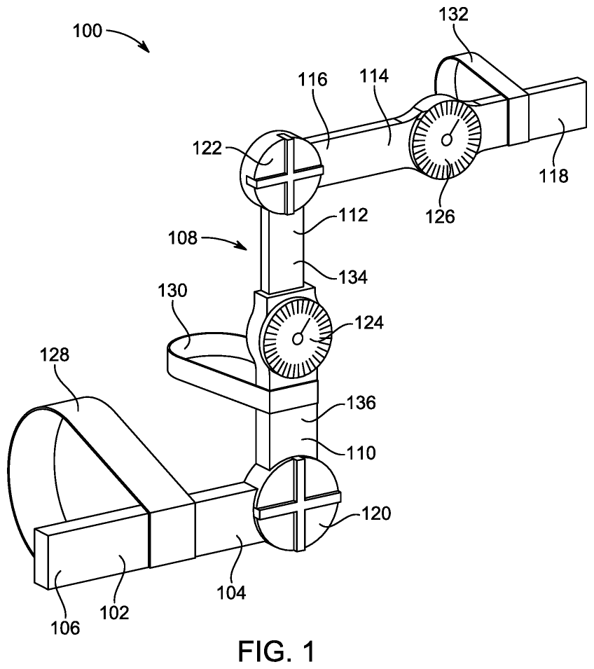

FIG. 1 illustrates a perspective view of a hip knee range of motion measurement device 100 (hereinafter referred to as “the device 100”). The device 100 includes a first movable arm 102 having a first end 104 and a second end 106, a second movable arm 108 having a first end 110 and a second end 112, and a third movable arm 114 having a first end 116 and a second end 118. In some embodiments, the length between the first end 104 on a far end of the first arm 102 and second end 106 on the other end of the first arm 102 is from 15 centimeters (cm) to 30 cm, preferably from 16 cm to 29 cm, preferably from 17 cm to 28 cm, preferably from 18 cm to 27 cm, preferably from 19 cm to 26 cm, preferably from 20 cm to 25 cm, or 22.5 cm. In some embodiments, the length from the first end 110 on a far end of second arm 108 and second end 112 on the other end of the second arm 108 is from 20 centimeters (cm) to 60 cm, preferably from 22.5 cm to 57.5 cm, preferably from 25 cm to 55 cm, preferably from 27.5 cm to 52.5 cm, preferably from 30 cm to 50 cm, preferably from 35 cm to 45 cm, or 40 cm. In some embodiments, the length between the first end 116 on a far end of the third arm 114 and second end 118 on the other end of the third arm 114 is from 20 centimeters (cm) to 60 cm, preferably from 22.5 cm to 57.5 cm, preferably from 25 cm to 55 cm, preferably from 27.5 cm to 52.5 cm, preferably from 30 cm to 50 cm, preferably from 35 cm to 45 cm, or 40 cm. In some embodiments, the first movable arm 102, second movable arm 108, and third movable arm 114 are fabricated of metal, plastic, ceramic, or a polymer. In some embodiments, the length of the second movable arm 108 is between 1.1 and 1.5 times greater than the length of both the first movable arm 102 and third movable arm 114, preferably between 1.2 and 1.4 times greater, or 1.3 times greater. The first movable arm 102, the second movable arm 108, and the third movable arm 114 are coplanar and are illustrated as rectangular blocks. However, this description for the purpose of illustration should not be construed as limited. In some embodiments, each of the first movable arm 102, the second movable arm 108, and the third movable arm 114 may include a circular cross-section, square cross-section, or polygonal cross-section. A first joint 120 of the device 100 is attached to the first end 104 of the first movable arm 102 and a second joint 122 is attached to the second end 112 of the second movable arm 108. The first and second joint 120 and 122 can be defined as a rotatable connector between the respective arms that gives the device 100 its flexibility in order for the movable arms to be coplanar. The first and second joints 120 and 122 are thus pivotally attached to each respective end of each respective movable arm. For example, the first joint 120 can rotate the first movable arm 102 around the first end 104 and the second movable arm 108 around the first end 110 so that each of the arms may be positioned in a coplanar fashion. Similarly, the second joint 122 can rotate the second movable arm 108 around the second end 112 and the third movable arm 114 around the first end 116 so that each of the arms may be positioned in a coplanar fashion. When the movable arms are coplanar due to the rotation of the first and second joints 120 and 122, the first and second joints 120 and 122 are also in the same plane as the movable arms. In some embodiments, the first joint 120 is substantially circular in shape and has a diameter ranging from 10 mm to 300 mm, preferably 50 mm to 250 mm, preferably 100 mm to 200 mm, or 150 mm. In some embodiments, the second joint 122 is substantially circular in shape has a diameter ranging from 10 mm to 300 mm, preferably 50 mm to 250 mm, preferably 100 mm to 200 mm, or 150 mm. In some embodiments, the first joint 120 has a diameter that is from 1.1 to 1.3 times greater than the diameter of the second joint 122, preferably between 1.125 and 1.275 times greater, preferably between 1.15 and 1.25 times greater, preferably between 1.175 and 1.225 times greater, or 1.2 times greater. The first joint 120 and second joint 122 are fabricated of plastic, metal, ceramic, polymers, or combination of the like. In some embodiments, the first and second joints 120 and 122 have a cross-shaped dial protruding from the front surface of the joint that allows the individual arms to be rotated more easily, as depicted in FIG. 1. The cross-shaped dial allows an assessor to determine the angle of slope with respect to an adjacent arm, for example ranges between 10° and 180° with respect to the adjacent arm. In a preferred embodiment, the dial determines the angle of slope in ranges between 10° and 90° based on realistic flexion of a human leg. The first end 110 of the second movable arm 108 is attached to the first joint 120 and the first end 116 of the third movable arm 114 is attached to the second joint 122. In alternate embodiments, the first joint 120 and second joint 122 are shaped like a diamond, triangle, pentagon, hexagon, circular, or any other polygonal shape.

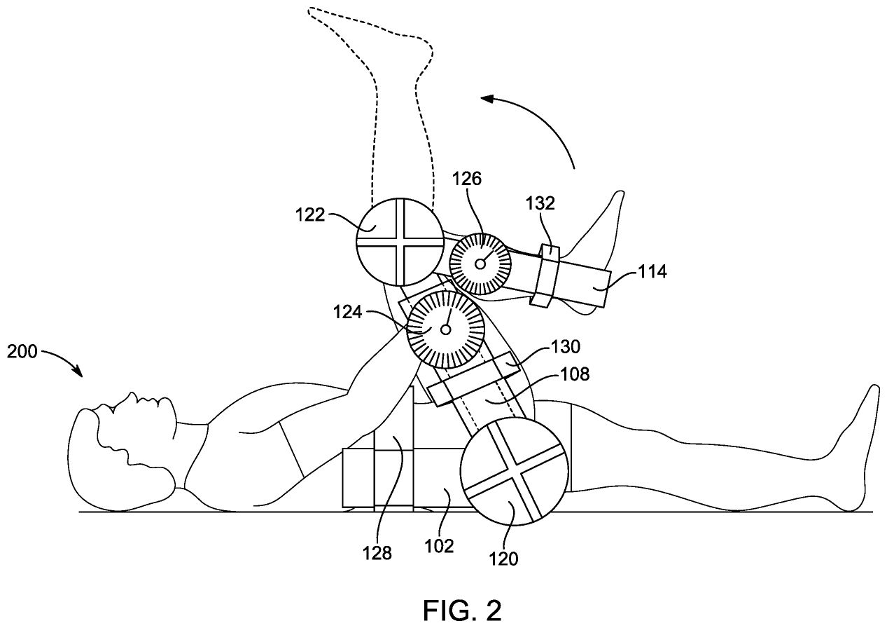

The device 100 also includes a first dial 124 attached to the second movable arm 108 and a second dial 126 attached to the third movable arm 114. The dials can be defined as a structure that is capable of measuring a slope, an elevation, or an angle of inclination of a specific axial axis of a body part or joint angle with respect to an adjacent arm. As seen in FIG. 2, the first dial 124 and second dial 126 can measure the angle of inclination of a clinical patient with respect to the adjacent arm, through the use of tick marks across an entire 360° circumference of the dial. For example, an angle of inclination is shown to be between the first arm 102 and second arm 108, with the stretching of the leg changing the angle between adjacent arms and the dials are configured record the angle. The first dial 124 and second dial 126 are mounted on each respective arm to clearly indicate the measure of inclination for the assessor. The first dial 124 and second dial 126 use the angle of inclination between adjacent arms as an input value to produce a voltage output. In some embodiments, the first dial 124 and second dial 126 are each an electrical resistive wheel that uses a photoelectric detector to determine how far each adjacent arm has spread apart to determine the angle of inclination as a signal input. The resistive wheel is fixedly attached on one arm and is electrically connected to the other arm to determine the distance each arm has spread with respect to the other arm. In some embodiments, the device 100 further includes an alarm which is activated if the joint angle exceeds a predetermined angle during the test. For instance, the first arm 102 is in the same plane as the ground in FIG. 2, but the second arm 108 can move independently from the first arm 102 to get a rough estimate of the angle between the adjacent arms.

A preferred embodiment of the invention utilizes gravity directed inclinometers for the dials each with a swinging pendulum mounted above a marked dial face, much like a compass needle mounted on a pin. In this embodiment each inclinometer face is permanently fixed onto an outer facing surface of the second and third arms coplanar to one another. A pendulum needle is loosely mounted at the center of the circular face of the inclinometer at a centrally disposed pin. Loose mounting of the pin permits the pin to swing and rotate around the pin. In this embodiment a physical therapist or medical practitioner can quickly and visually determine an angle of inclination based on visual inspection of the positions of the needles of the first and second dials relative to markings on the faces of the inclinometer faces. The face of each dial has a commonly oriented reference point permitting mathematical manual calculation of angles.

In some embodiments, the first dial 124 and second dial 126 are substantially circular in shape and have diameters ranging from 10 mm to 300 mm, preferably 50 mm to 250 mm, preferably 100 mm to 200 mm, or 150 mm. In some embodiments, the first dial 124 and second dial 126 contain at least 20 tick marks to more easily measure the full 360° range of motion, preferably 30 tick marks, preferably 40 tick marks, or 60 tick marks. In some embodiments, the first dial 124 is equally spaced between the first end 110 and the second end 112 of the second movable arm 108, and the second dial 126 is equally spaced between the first end 116 and the second end 118 of the third movable arm 114. In some embodiments, the first dial 124 is equally spaced between the first end 110 and second end 112 of the second movable arm 108 by 50 millimeters (mm) to 800 mm, preferably between 100 mm and 700 mm, preferably between 200 mm and 600 mm, preferably between 300 mm and 500 mm, or 400 mm. In some embodiments, the second dial 126 is equally spaced between the first end 116 and second end 118 of the third movable arm 114 by 50 millimeters (mm) to 800 mm, preferably between 100 mm and 700 mm, preferably between 200 mm and 600 mm, preferably between 300 mm and 500 mm, or 400 mm. In some embodiments, a sensor is attached to each of the first dial 124 and the second dial 126 to measure a range of motion more precisely. In some embodiments, the sensors are optical sensors and can include optical encoders. In some embodiments, the first dial 124 and second dial 126 are fabricated of plastic, metal, ceramic, polymers, or combination of the like. In some embodiments, the first dial 124 and the second dial 126 are digital and can monitor the slope, elevation or tilt of a clinical patient with respect to the adjacent arm. In some embodiments, the first dial 124 and second dial 126 are digital inclinometers used to measure the angle of slope with respect to the adjacent arm. In some embodiments, the first dial 124 and second dial 126 are fixedly attached to their respective movable arms so as to read the measure of inclination more precisely. A first movable strap 128 is attached to the first movable arm 102, a second movable strap 130 is attached to the second movable arm 108, and a third movable strap 132 is attached to the third movable arm 114. In some embodiments, the first strap 128, second strap 130, and third strap 132 are fabricated of Velcro, wool, felt, nylon, velvet, knit fabrics, or the like. In some embodiments, the attachment of the first strap 128 to the first movable arm 102, second strap 130 to the second movable arm 108, and third strap 132 to the third movable arm 114 to the clinical patient creates a circular cross-section around the respective body part of the patient, with a diameter ranging from 100 mm to 1000 mm, preferably 200 mm to 900 mm, preferably 300 mm to 800 mm, preferably 400 mm to 700 mm, preferably 500 mm to 600 mm, or 550 mm.

FIG. 2 illustrates a clinical patient 200 wearing the device 100 for physical therapy. In a preferred embodiment, the device 100 is configured to provide a range of motion values of both a hip and a knee of the clinical patient 200 simultaneously. In some embodiments, the first movable arm 102 is configured to remain longitudinally oriented with a horizontal axis of a pelvis of the clinical patient 200 with the first movable strap 128 located around a waist of the clinical patient 200, the second movable arm 108 is configured to in axial alignment to a horizontal axis of a femur of the clinical patient 200 with the second movable strap 130 located around the thigh of the clinical patient 200, and the third movable arm 114 is configured to remain in axial alignment to a horizontal axis of a tibia of the clinical patient 200 with the third movable strap 132 located around an ankle portion of the clinical patient 200. In some embodiments, each of the first movable strap 128, the second movable strap 130, and the third movable strap 132 may be a non-continuous band and may include a clip (not shown) to join ends of the of the non-continuous band. In an example, the clip may be a buckle. Such arrangement may allow the clinical patient 200 to easily wrap the respective movable strap around corresponding portions of the body as mentioned above and tighten the movable strap to a desired degree of comfort. In some embodiments, each of the first movable strap 128, the second movable strap 130, and the third movable strap 132 may be made of an elastic material. In some embodiments, each of the first movable strap 128, the second movable strap 130, and the third movable strap 132 may be moved along a length of respective movable arms. In some embodiments, each of the first movable strap 128, the second movable strap 130, and the third movable strap 132 may be detachably coupled to the respective movable arms. In such cases, each of the movable straps may include an indicator, such a text or symbol indication portion of the body, to help the clinical patient 200 to correctly locate the movable strap with the respective movable arm.

In a preferred embodiment the first movable strap 128 is made of a flexible thermoplastic material such as PVC and has a thickness of 0.1-0.4 cm to provide flexion of the strap. In this preferred embodiment the first movable strap 128 is mounted in a generally perpendicular orientation to a plane defined by the first, second and third movable arms. The first movable strap 128 is sized to curl around the midriff or abdomen of a patient undergoing analysis with the device 100. In this configuration the first movable strap 128 is releasably attachable to the first arm 102 so that straps of different size may be mounted on the first movable arm 102 depending on the size, physique and/or physical features of the patient. In some embodiments, the first movable strap 128 can be a brace, a stay, a sleeve, a sling, a garment, a wrap, or a strap. The first movable strap 128 is fitted around the abdomen of the clinical patient in order to effectively lay the patient as flat on the horizontal ground as possible with a single assessor.

In a preferred embodiment the second movable strap 130 is made of a flexible thermoplastic material such as PVC and has a thickness of 0.1-0.4 cm to provide flexion of the strap. In this preferred embodiment the second movable strap 130 is mounted in a generally perpendicular orientation to a plane defined by the first, second and third movable arms. The second movable strap 130 is sized to curl around the upper thigh or hip joint region of a patient undergoing analysis with the device 100. In this configuration the second movable strap 130 is releasably attachable to the second arm 108 so that straps of different size may be mounted on the second movable arm 108 depending on the size, physique and/or physical features of the patient. In some embodiments, the second movable strap 130 can be a brace, a stay, a sleeve, a sling, a garment, a wrap, or a strap. The second movable strap 130 is fitted around the hip joint in order to effectively stretch the hamstring of the clinical patient with respect to the adjacent arm as possible with a single assessor to better quantify hamstring flexibility while keeping the patient's hip stationary.

In a preferred embodiment the third movable strap 132 is made of a flexible thermoplastic material such as PVC and has a thickness of 0.1-0.4 cm to provide flexion of the strap. In this preferred embodiment the third movable strap 132 is mounted in a generally perpendicular orientation to a plane defined by the first, second and third movable arms. The third movable strap 132 is sized to curl around the ankle or shin region of a patient undergoing analysis with the device 100. In this configuration the third movable strap 132 is releasably attachable to the third arm 114 so that straps of different size may be mounted on the third movable arm 114 depending on the size, physique and/or physical features of the patient. In some embodiments, the third movable strap 132 can be a brace, a stay, a sleeve, a sling, a garment, a wrap, or a strap. The third movable strap 132 is fitted around the ankle joint in order to effectively stretch the hamstring of the clinical patient with respect to the adjacent arm as possible with a single assessor and to extend the second and third arm in a coplanar fashion when the patient stretches their leg in order to more effectively measure hamstring flexibility by a single assessor.

When the device 100 is worn by the clinical patient 200, various parts of the device 100 may function as indicators to correctly position the device 100 along the body. For example, the first joint 120 is configured to be mounted adjacent to a hip joint of the clinical patient 200 and is configured to remain longitudinally oriented with a horizontal axis of a hip of the clinical patient 200. Additionally, the second joint 122 is configured to be mounted adjacent to a knee joint of the clinical patient 200. As such, until the first joint 120 and the second joint 122 are positioned properly along the body, the clinical patient 200 or a helper to the clinical patient 200 may adjust the device 100 accordingly. In some embodiments, the first joint 120 and the second joint 122 include adjustable breaks. In some embodiments, the adjustable breaks are locks in which the respective joint can be stabilized at a predetermined angle. For example, to measure the popliteal angle of the knee during the AKE test, the hip joint can be stabilized at 90 degrees by using the adjustable locks to secure the hip joint at 90 degrees within the device 100. In some embodiments, a length of the first movable arm 102, a length of the second movable arm 108, and a length of the third movable arm 114 are each slidably adjustable by half the total length of the first movable arm 102, the second movable arm 108, and the third movable arm respectfully 114. In some embodiments, the length of the first movable arm 102 is slidably adjustable to between 0.3 and 0.7 times a total length of the first movable arm 102, preferably between 0.4 and 0.6, or most preferably 0.5 times the length. In some embodiments, the length of the second movable arm 108 is slidably adjustable to between 0.3 and 0.7 times a total length of the second movable arm 108, preferably between 0.4 and 0.6, or most preferably 0.5 times the length. In some embodiments, the length of the third movable arm 114 is slidably adjustable to between 0.3 and 0.7 times a total length of the third movable arm 114, preferably between 0.4 and 0.6, or most preferably 0.5 times the length. In some embodiments, as illustrated in FIG. 1, the second movable arm 108 may include a telescopic arrangement, where a sliding portion 134 may be configured to slide along a length of a receiving portion 136 of the second movable arm 108. In some embodiments, the telescopic arrangement is configured so that the sliding portion 134 slides along the second movable arm between 0.4 and 0.9 times the length of the receiving portion 136, preferably between 0.5 and 0.8, preferably between 0.6 and 0.7, or 0.65 times the length of the second movable arm 108. Such arrangement helps to adjust the device 100 according to the length of the thigh of the clinical patient 200. In some embodiments, the second movable arm 108 may include a locking arrangement (not shown) to lock the movement of the sliding portion 134 with respect to the receiving portion 136.

The first dial 124 and the second dial 126 are configured to measure a joint angle of the clinical patient 200. The first dial 124 is configured to measure hip angle of the clinical patient 200 at the hip joint angles ranging from 0 degrees to 360 degrees, preferably from 30 to 330 degrees, preferably from 60 to 300 degrees, preferably from 90 to 270 degrees, preferably from 120 degrees to 240 degrees, preferably from 150 degrees to 210 degrees, or 180 degrees and the second dial 126 is configured to measure a popliteal angle of the clinical patient 200 at the knee joint angles ranging from 0 degrees to 360 degrees, preferably from 30 to 330 degrees, preferably from 60 to 300 degrees, preferably from 90 to 270 degrees, preferably from 120 degrees to 240 degrees, preferably from 150 degrees to 210 degrees, or 180 degrees. Further, the second movable arm 108 is adjustable in a range of from 0 degrees to 360 degrees, preferably from 30 to 330 degrees, preferably from 60 to 300 degrees, preferably from 90 to 270 degrees, preferably from 120 degrees to 240 degrees, preferably from 150 degrees to 210 degrees, or 180 degrees. Additionally, in some embodiments, each of the first joint 120 and the second joint 122 is rotatable 360 degrees.

With such arrangement, the first movable arm 102, the second movable arm 108, and the third movable arm 114 are configured to stabilize the hip joint of the clinical patient 200 while measuring the popliteal angle, or angle of knee flexion, of the clinical patient 200.

To this end, the present disclosure provides the device 100 that addresses challenges of conventional joint angle measurement methods and helps clinicians, and physical therapists obtain accurate measurements of the popliteal angle. The device 100 is small, lightweight, portable, and inexpensive. In some embodiments, the device 100 weighs between 2 and 20 pounds, preferably between 4 and 18 pounds, preferably between 6 and 16 pounds, preferably between 8 and 14 pounds, or 10 pounds. The device 100 also measures hamstring length and flexibility (active knee extension test and passive knee extension test) accurately by a single assessor with hip stabilized in a certain degree. In addition, the device 100 aids in measuring the popliteal angle at various degrees of hip joint angles, which is often required in the clinical population. Physical therapists may easily train the hip joint at various positions of the knee joint with the help of the device 100. Although the device 100 herein has been described with respect to the clinical patient 200, it should be understood that the device 100 may be used by a person who is involved in athletic and need to improve strength in the legs.

As used herein, the terms “a” and “an” and the like carry the meaning of “one or more.”

Obviously, numerous modifications and variations of the present invention are possible in light of the above teachings. It is, therefore, to be understood that, within the scope of the appended claims, the invention may be practiced otherwise than as specifically described herein.

1. A hip knee range of motion measurement device for physical therapy, comprising:
a first movable arm having first and second ends;
a first joint attached to the first end of the first movable arm;
a second movable arm having first and second ends, wherein the first end of the second movable arm is attached to the first joint;
a first dial attached to the second movable arm;
a second joint attached to the second end of the second movable arm;
a third movable arm having first and second ends, wherein the first end of the third movable arm is attached to the second joint;
a second dial attached to the third movable arm; and
a first movable strap attached to the first movable arm, a second movable strap attached to the second movable arm, and a third movable strap attached to the third movable arm; wherein
the first movable arm is configured to remain longitudinally oriented with a horizontal axis of a pelvis of a clinical patient with the first movable strap, the second movable arm is configured to remain in axial alignment to a horizontal axis of a femur of the clinical patient with the second movable strap, and the third movable arm is configured to remain in axial alignment to a horizontal axis of a tibia of the clinical patient with the third movable strap; and
the first joint is configured to be positioned adjacent to a hip joint of the clinical patient and is configured to remain longitudinally oriented with a horizontal axis of a hip of the clinical patient; and
the second joint is configured to be positioned adjacent to a knee joint of the clinical patient; and
the first and second dials are configured to measure a joint angle of the clinical patient; and
the first, second, and third movable arms are coplanar.

2. The device of claim 1, wherein the device is configured to provide range of motion values of both the hip and a knee of the clinical patient simultaneously.

3. The device of claim 1, wherein the first, second, and third movable arms are configured to stabilize the hip joint of the clinical patient while measuring a popliteal angle of the clinical patient.

4. The device of claim 1, wherein the first dial is configured to measure the hip angle of the clinical patient at hip joint angles ranging from 0 degrees to 360 degrees.

5. The device of claim 1, wherein the second dial is configured to measure a popliteal angle of the clinical patient at the knee joint ranging from 0 degrees to 360 degrees.

6. The device of claim 1, wherein the first and second joint comprise adjustable breaks.

7. The device of claim 1, wherein the first dial is equally spaced between the first end and the second end of the second movable arm.

8. The device of claim 1, the second dial is equally spaced between the first end and the second end of the third movable arm.

9. The device of claim 1, wherein the second movable arm is configured to be adjustable in a range of from 0 degrees to 360 degrees.

10. The device of claim 1, wherein the first joint is rotatable 360 degrees.

11. The device of claim 1, wherein the second joint is rotatable 360 degrees.

12. The device of claim 1, wherein a length of the first movable arm, a length of the second movable arm, and a length of the third movable arm are each slidably adjustable by half the total length of the first movable arm, the second movable arm, and the third movable arm respectfully.
